(19) United States
(12) Patent Application Publication
(10) Pub. No.: US 2007/0212786 A1
(43) Pub. Date: Sep. 13, 2007

(54) Monitoring heparin by microelectronic devices

(76) Inventors: Scott Manalis, Cambridge, MA (US); Tzu Liang Loh, Watertown, MA (US); Michel Godin, Somerville, MA (US); Nebojsa M. Milovic, Basel (CH); Jonathan R. Behr, Cambridge, MA (US); Aarthi Chandrasekaran, Cambridge, MA (US); Ram Sasisekharan, Cambridge, MA (US)

Correspondence Address: CHOATE, HALL & STEWART LLP, TWO INTERNATIONAL PLACE, BOSTON, MA, 02110, US

(21) Appl. No.: 11/540,695

(22) Filed: Sep. 29, 2006

Related U.S. Application Data
(60) Provisional application No. 60/722,023, filed on Sep. 29, 2005.

(51) Int. Cl. G01N 33/86

(52) U.S. Cl. 436/69

(57) ABSTRACT

In one aspect, the present invention provides a device and method for real-time, direct detection of heparin in buffer and in serum comprising a microfluidic field-effect device as an affinity biosensor. The sensor is based on an electrolyte-insulator-silicon structure, and is manufactured by a standard high-yield silicon microfabrication process. The binding of heparin to the sensor surface induces a change in the insulator-electrolyte surface potential, which is measured as a change in sensor capacitance. To ensure the binding selectivity, protamine and antithrombin III are used as affinity probes.

### PRIORITY INFORMATION

This application claims priority to U.S. Provisional Application No. 60/722,023, filed Sep. 29, 2005.

### BACKGROUND OF THE INVENTION

Heparin has been used clinically as an anticoagulent for over 60 years, and it is second to insulin as a natural therapeutic agent. Other biological activities of heparin include release of lipoprotein lipase and hepatic lipase, inhibition of complement activation, inhibition of angiogenesis and tumor growth, and antiviral activity. The biological activities of heparin result from its interaction with proteins, the most well-characterized being its interaction with antithrombin III (ATIII), a serine protease inhibitor that mediates the anticoagulant activity of heparin.

In a clinical setting, it is critical to maintain heparin levels that are sufficient to prevent thrombosis but avoid risks of bleeding. Considering that more than half a billion doses of heparin are used annually, there have been intensive efforts to develop simple sensor systems that could detect heparin directly in blood or serum samples. The widely used clinical procedures for monitoring heparin anticoagulant activity are measurements of activated clotting time (ACT) and activated partial thromboplastin time (APTT). However, these methods do not assess the actual heparin concentration. These procedures are based on the time required for clot formation upon contact activation with an agent such as kaolin, and the heparin level is correlated to the delay in the appearance of a clot. Although these methods have been used for a long time, the ACT value is not an accurate indicator of blood heparin levels since the clotting time can also be affected by other factors, such as hypothermia or hemodilution (commonly encountered during surgery), abnormal levels of AT-III, and other clotting factors. Furthermore, these existing methods for actual determination of heparin concentrations are indirect and include protamine titration and colorimetric assay of anti-Xa activity, which is unsuitable for nontransparent samples like blood.

Advancements in the understanding of the important biological role of saccharides and their interactions with proteins depend on the development of bioanalytical methods. Both synthesis and analysis of saccharides are hampered by their complex molecular structures, intrinsic heterogeneity of samples, and difficulty of characterization and detection. Several methods of heparin detection have been described in literature. However, practical commercial and mass use of heparin biosensors is limited by the requirement to use additional reagents and/or specialized laboratory equipment. For example, the monitoring of heparin has been reported during cardiopulmonary bypass surgery and other invasive procedures. Binding of fibroblast growth factor (FGF) to specific heparin sequences was analyzed by using a radioactive-labeling technique. SPR has been used as a sensor technique for heparin detection and analysis. QMC was also applied for heparin detection. Ion-channel sensor methods were shown to detect heparin, however these methods exhibited a decrease in reproducibility and precision of the determined concentrations after repeated use of the electrode. The 2002 Analytica chimica acta QCM study has been limited to PBS only. The 2005 Anal. Biochem. spectrofotometric (using tetracycline-europium probe) study admits serious interference from serum albumin in their whole-blood measurements. There remains a need to develop direct and sensitive methods for the measurement and control of heparin levels.

### DEFINITIONS

In accordance with the present invention and as used herein, the following terms, are defined with the following meanings, unless explicitly stated otherwise.

As used herein, the terms “microfluidic,” “microchannel,” and “microfluidic channel” refers to a structure or channel having at least one dimension that may most conveniently be expressed in terms of micrometers. For example, the term “microfluidic channel” may refer to a channel having at least one dimension of approximately 500 μm or less, approximately 100 μm or less, approximately 50 μm or less, approximately 20-50 μm, approximately 10-20 μm, approximately 5-10 μm, approximately 1-5 μm, approximately 1 μm, or between 0.1 and 1 μm. One or ordinary skill in the art will recognize that the dimensions of such channels may run into the millimeters, but that most dimensions are in the micrometer range.

Certain compounds disclosed in the present invention, and definitions of specific functional groups are also described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. Furthermore, this invention is not intended to be limited in any manner by the permissible substituents of organic compounds. Combinations of substituents and variables envisioned by this invention are preferably those that result in the formation of stable compounds. The term “stable,”, as used herein, preferably refers to compounds which possess stability sufficient to allow manufacture and which maintain the integrity of the compound for a sufficient period of time to be detected and preferably for a sufficient period of time to be useful for the purposes detailed herein.

### BRIEF DESCRIPTION OF THE FIGURE

FIG. 1A is a microscopic image of a field-effect device containing two sensing surfaces and overlaid by a PDMS slab that forms a microfluidic channel according to one embodiment of the present invention;

FIG. 1B is a schematic illustration of device operation according to another embodiment of the invention, showing the depleted region under the sensor surface;

FIG. 1C illustrates the principle of differential measurement according to one embodiment of the invention;

FIG. 1D graphically demonstrates the sensitivity of surface-unmodified field-effect sensor according to one embodiment of the invention;

FIG. 2A is a schematic illustration of the response to 5 U/ml heparin solution for active sensor and control sensor and the differetial measurement for a protamine field effect sensor according to an embodiment of the invention (the inset illustrates the principle of surface immobilization by physisorption);

FIG. 2B shows a dose-response curve for a protamine sensor according to another embodiment of the invention;

FIG. 3A illustrates a dose-response curve for heparin measurements in human blood serum using a protamine sensor for a broad range according to one embodiment of the invention;

FIG. 3B illustrates a dose-response curve for heparin measurements in human blood serum using a protamine sensor for a clinically relevant linear range according to one embodiment of the present invention;

FIG. 4A illustrates the strategy for surface immobilization of heparin receptors and heparin binding according to certain embodiments of the invention;

FIG. 4B shows a dose-response curve for the ATIII sensor with unfractionated heparin, and with chondroitin sulfate, according to yet another embodiment of the invention;

FIG. 4C shows a dose-response curve for the ATIII sensor with Arixtra®, and desulfated Arixtra, according to other embodiments of the invention (solid lines present the result of fitting to Langmuir isotherm whereas dashed lines serve to connect the data points); and

FIG. 5 depicts a structure of heparin.

### DETAILED DESCRIPTION OF CERTAIN PREFERRED EMBODIMENTS OF THE INVENTION

The methods and devices of the present invention can combine biology, chemistry, and physics and engineering to detect and monitor biomolecules. For example, biology is utilized as a recognition system, chemistry for surface functionalization, and physics and engineering for transducers and/or instrumentation to pick up and/or analyze a signal.

Heparin is currently one of the most essential and powerful anticoagulants, and the most widely used drug for the prevention of blood clotting. Monitoring heparin levels in blood is vital during and after surgeries, and therefore it is essential to enable real-time detection and measurement. Current methods to monitor heparin are indirect, slow, nonspecific, and sometimes unreliable.

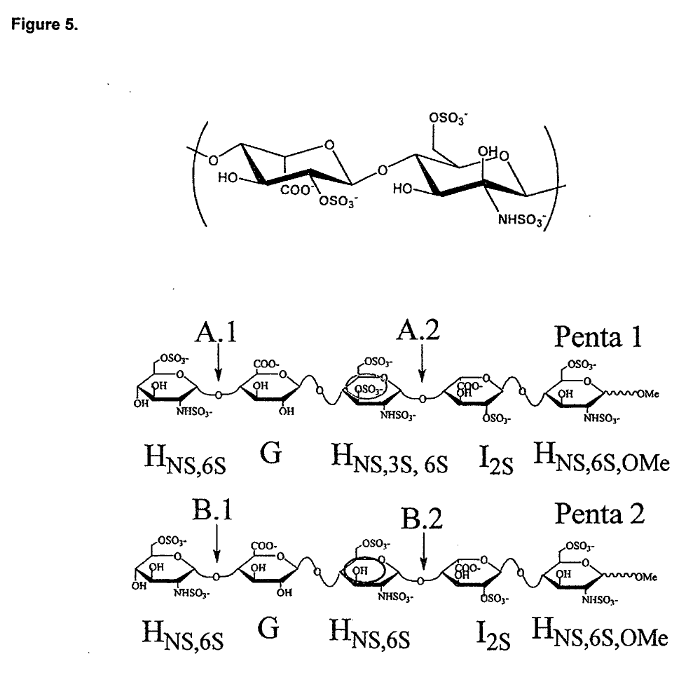

Heparin is a linear polysaccharide consisting of uronic acid-(1→4)-D-glucosamine repeating disaccharide subunits. The disaccharide subunits are heavily N-sulfate, O-sulfate and N-acetyl groups bring an overall high negative charge. Variable patterns of substitution of the disaccharide subunits with N-sulfate, O-sulfate and N-acetyl groups give rise to a large number of complex sequences. (See, e.g., FIG. 5).

As discussed above, there remains a need to develop direct and sensitive methods for the measurement and control of heparin levels. In one aspect, the present invention provides a method and device for real-time, label-free, direct detection of heparin in serum by its highly negative intrinsic charge. Label-free electronic detection has significant advantages over label-dependent detection. For example, a fluorescence detector has high sensitivity, but requires sample labeling and optical readout. Electronic detection however, does not require sample pre-treatment, facilitates reduced possessing time and costs, and has an ease of integration and multiplexing.

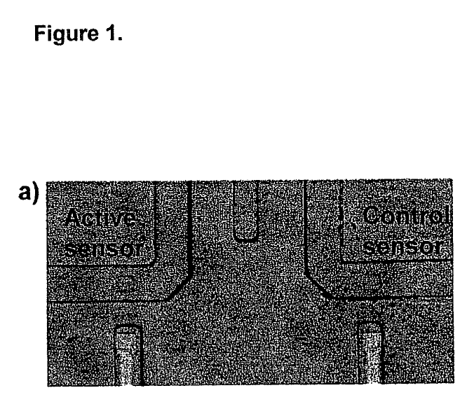

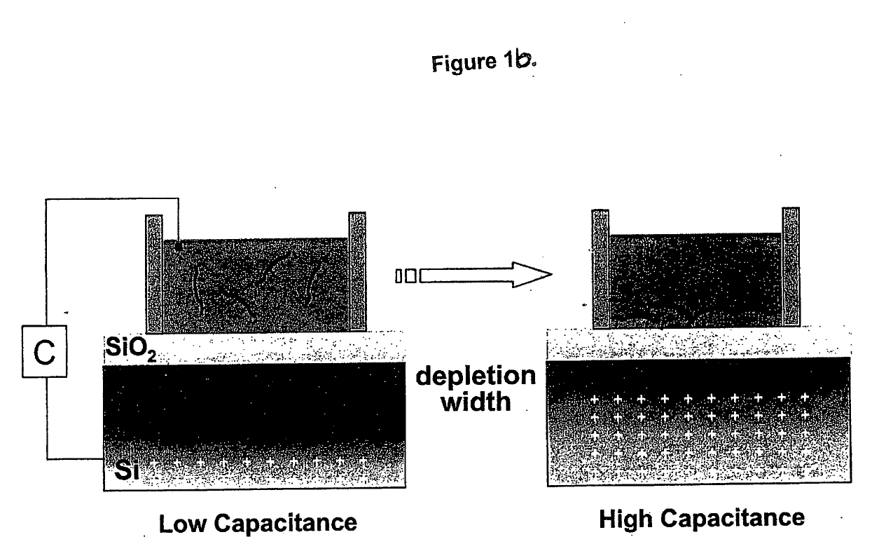

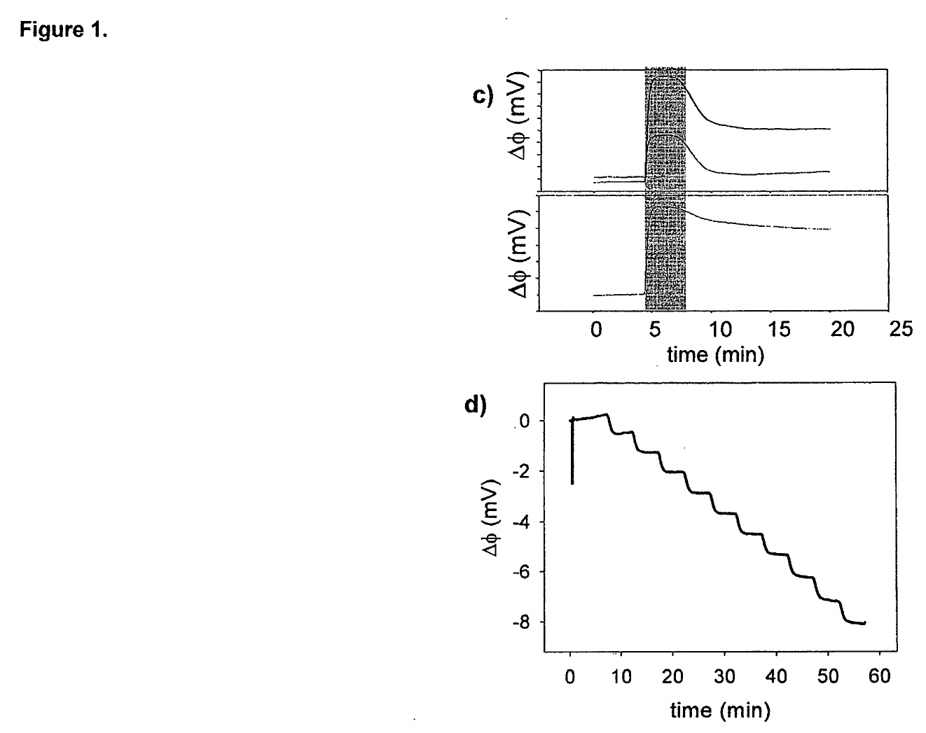

In another aspect of the invention, a microfluidic field-effect sensor is used to monitor heparin levels. FIG. 1A is a microscopic image of a field-effect device according to one embodiment of the invention. The field-effect sensor consists of an electrolyte-insulator-silicon (EIS) structure. The EIS structure may be manufactured by a standard high-yield silicon microfabrication process. In various embodiments, the binding of heparin to the sensor surface alters the insulator-electrolyte surface potential, which is detected by measuring the EIS capacitance. In the embodiment depicted in FIG. 1A, the device contains two 50×50 nm sensing surfaces and is overlaid by a PDMS slab that forms a microfluidic channel. With this configuration, the sensor surfaces are individually functionalized for differential detection and the sample is subsequently delivered to both sensors. The dual sensor set-up allows for experiments to be run on an “active” sensor and compared against a “control” sensor (as described below). In other embodiments, a PDMS slab containing a single channel common for both sensors may be used.

The device further comprises a liquid delivery system. The sensor exposure to the analyte is controlled by adjusting the flow rate and injection volume of the analyte through the liquid delivery system.

The operating principle of the field-effect measurement is shown schematically in FIG. 1B. When charged molecules absorb near the sensor surface, the surface potential at the insulator-electrolyte interface is changed and this alters the depth of the carrier depletion region in the underlying silicon. The depletion depth may be continuously monitored by measuring the current through the sensor. FIG. 1C illustrates the principle of differential measurement according to one embodiment of the invention. FIG. 1D graphically demonstrates the sensitivity of a surface-unmodified field-effect sensor according to another embodiment of the invention. FIG. 1D shows the change of surface potential versus the change in pH ranging from 7.00 to 6.80 in ten 0.02 pH change increments. The spike at 0 minutes corresponds to the externally applied potential change of 2.5 mV, to which surface potential measurements are normalized.

Specificity towards a target biomolecule is achieved by functionalizing the sensor surface with receptors that are typically a biological partner of the target. In one embodiment, differential pairs of sensors may be used, to ensure sensor selectivity and eliminate the effects of unwanted interference arising from non-specific binding and solution conditions (e.g., pH and ionic strength). For example, high selectivity in buffer and in human serum may be achieved by using heparin's physiological partner's protamine or antithrombin III as affinity probes and an additional surface passivated sensor to create a differential measurement.

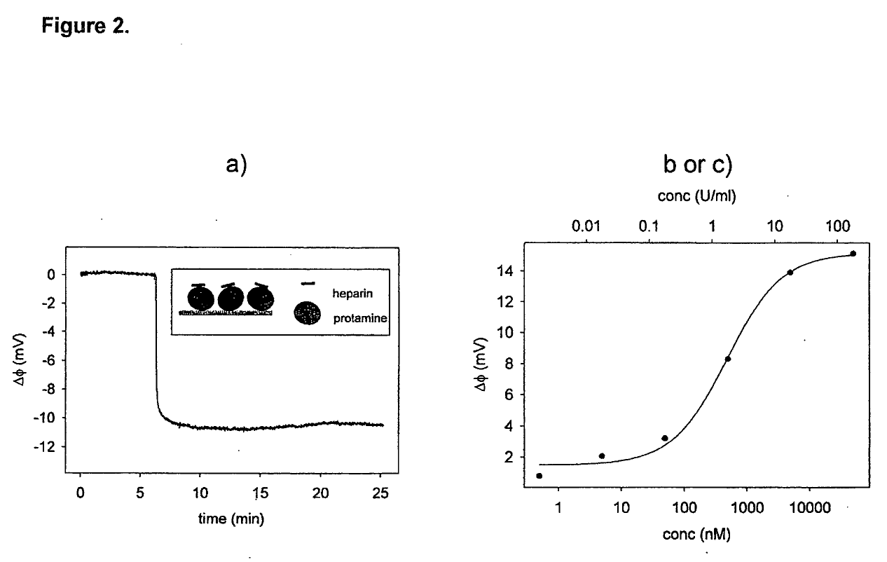

Protamine is a 5 kD protein with high affinity to heparin due to electrostatic interactions between its multiple arginine residues with anionic site in heparin. This high affinity to heparin makes protamine therapeutically useful for neutralizing heparin activity in vivo. In one example, the “active” sensor was surface modified with the actual receptor, and the “control” sensor surface was “passivated” with BSA which is a known non-binder to the analyte. The signals for both sensing surfaces were simultaneously measured, and the signal of the active sensor was subtracted from that of the control in order to reject the common mode signal. Protamine was immobilized to the sensor surface by a 10 minute exposure to a 20.0 μM protamine solution. The change of the surface potential was monitored during this process, and the result is shown in FIG. 2A. Upon protamine injections, the surface potential dropped by 12.×mV, and the baseline remained at the same level upon the reintroduction of the buffer. The decrease in the baseline level is consistent with the cationic molecular charge of protamine at neutral pH. Repeated protamine injections yielded no further decrease of the baseline level, indicating completed surface saturation.

FIG. 2B exemplifies a sensor response to protamine in one embodiment of the invention, wherein heparin was injected at a clinically relevant concentration of 5 U/ml. The signal of the active protamine sensor increased during the injection, and the baseline remained elevated following the buffer rinse. At the same time, the signal control sensor, passivated by BSA also responded to the heparin injection, but only transiently since the original baseline remained upon the buffer rinse, consistent with no heparin binding. The transient increase of signal for the control sensor can be attributed to the slight difference in the solution conditions, i.e., pH, temperature, and ionic strength between the running buffer and the sample, as well as non-specific binding. The resulting differential signal removed the unwanted artifacts and provided an exemplary response to heparin binding of the active protamine sensor.

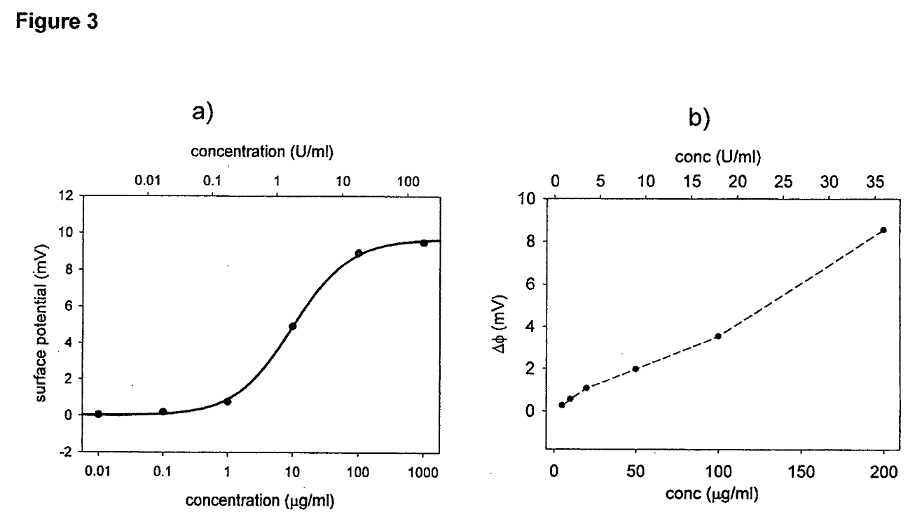

FIG. 3A and FIG. 3B illustrate a dose response curve for heparin in human serum according to another embodiment of the invention. The figures illustrate that although serum is a complex mixture of biomolecules, the performance of the sensor is relatively unaffected.

The physiological role of heparin for controlling blood coagulation is to bind to AT-III which is a major inhibitor of the coagulation cascade. Upon binding to a specific pentasaccharide sequence within a heparin model, AT-III undergoes a major conformational change. The resulting AT-III-heparin complex acts as a rapid, potent inhibitor of coagulation factors such as thrombin and factor Xa. Heparin structure is heterogeneous (Mw ranging from 3 to 30 KD and activity ranging from x to y U/mg) because the specific physiologically active pentasaccharide unit is variable distributed along the molecule sequence. Moreover, heparin is degraded in vivo by a set of sequence-specific hydrolytic enzymes. In various embodiments of the invention, the level of clinically-relevant active heparin is determined, rather than the total heparin concentration.

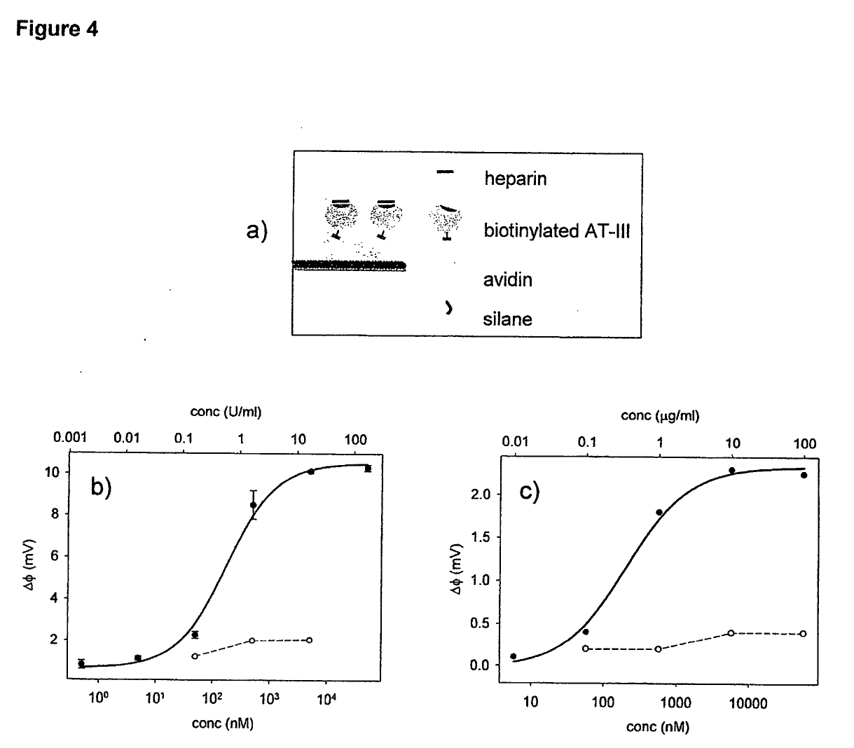

In one embodiment, AT-III is used as a covalently immobilized surface receptor. In this embodiment, the present invention can monitor active heparin. FIG. 4A illustrates the surface immobilization chemistry according to this embodiment. The surface immobilization chemistry involves aldehyde-terminated silanization of the SiO₂ surface, followed by the reductive amination of the aldehyde groups to the surface-exposed amino groups of avidin, blocking the unreacted sites by ethanolamine, and attachment of covalently-immobilized avidin to biotinylated AT-III. Prior to biotinylation, the active sites of AT-III are reversibly blocked and thus protected from reacting with the biotinylation reagent. Upon deblocking, the resulting bAT-III remains fully active because the heparin-binding site remains intact and unhindered because the introduced biotin groups are positioned away from it. This immobilization strategy ensures full activity and the desired surface orientation of AT-III for maximizing the sensor performance. FIG. 4B shows the dose-response curve for the AT-III sensor for heparin as described in the embodiment above. The selectivity of the AT-III sensor to heparin sequence was measured by examining the binding affinity to chondroitin sulfate, a negatively charged polysaccharide structurally related to heparin. The response of the AT-III sensor to chondroitin sulfate is neglible as show in FIG. 4b. The ability of the AT-III sensor to discriminate heparin from other similar polyanionic biomolecules is well suited for measurement in real-life samples.

FIG. 4C shows a dose response curve to Arixtra for the AT-III sensor. Arixtra is a pentasaccharide containing the actual sequence involved in physiologically relevant AT-III binding. The dose response curve shows accurate and sensitive direct detection of the present invention to low molecular weight heparins (LMWH). LMWHs have been used for prophylaxys of deep venous thromboembolysms, with occurrence as high as 50% in patients undergoing elective surgical procedures. Unlike unfractionated heparin, the LMWH has a long elimination half-time and low incidence of hemorrhage. The presence of LMWH in blood does not significantly affect the clotting time in broadly used ACT and APTT tests which makes these methods unsuitable for measuring LMWH in vivo. The remaining colorimetric anti Xa assay is limited to transparent samples, such as diluted plasma, and is therefore incompatible with whole blood measurements.

Dose-response curves acquired in the examples above revealed a detection limit of less than 0.01 U/ml, which is an order of magnitude lower than clinically relevant concentrations and superior to existing reported methods. In various embodiments, the present invention directly measures heparin concentration in the range of 0.01 U/mL to 10 U/mL (to achieve a 10× improvement in sensitivity). In another embodiment, the device comprises a thin insulator (e.g., <2 nm) at the sensor surface that captures heparin in close proximity to the field-sensitive silicon while high selectivity is achieved through a differential configuration that eliminates unwanted signals resulting from electronic disturbances, signal drift, variation in temperature, pH, ionic strength, and from non-specific binding. The device may be batch-fabricated by well-established silicon microfabrication processing and integrated with conventional PDMS or glass microfluidics.

The methods and devices of the present invention may be integrated with conventional fluidic delivery systems used for standard clinical blood analysis instrumentation. The results shown indicate that various embodiments of the present invention may be used as a bedside clinical device for continuous monitoring and maintenance of therapeutic levels of heparin and heparin-based oligosaccharide drugs. The present invention could be, for example, implemented within the extracorporeal fluidic system and integrated with other sensors for in-vivo measurements during surgery, or used as a home device during the patient's recovery.

### Equivalents

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### Exemplification

The method of this invention can be understood further by the examples that illustrate some of the processes by which the inventive method may be practiced. It will be appreciated, however, that these examples do not limit the invention. Variations of the invention, now known or further developed, are considered to fall within the scope of the present invention as described herein and as hereinafter claimed.

Device Design and Packaging

Field effect sensor chips based on the EIS structure were fabricated on six inch wafers using standard processes at the MIT Microsystems Technology Laboratory. Field sensitive regions ranging from 50×50 μm to 80×80 μm were defined by p-type doping and electrically isolated by the n-type substrate. Metal contact pads were connected to the field sensitive regions by heavily doped p-type traces. The n-type and heavily doped p-type regions were passivated with 1 um of silicon nitride that was deposited by low pressure chemical vapor deposition (LPCVD). The silicon nitride was removed over the field sensitive region which then passivated by native silicon oxide. Reference electrodes were defined adjacent to the field-sensitive region by evaporating 10 nm of chromium and 1 um of gold directly on the heavily doped p-type trace.

Prior to surface functionalization, the sensor chip was cleaned by (i) acetone rinse followed by a 5 minute sonication, (ii) immersion in a freshly prepared mixture of sulfuric acid and hydrogen peroxide (2:1 v/v), and (iii) oxygen plasma treatment for 60 seconds, ca. 15 W. Microfluidic channels were immediately placed over the field sensitive regions by overlaying a patterned PDMS slab. The PDMS slab containing 100 μm-wide microfluidic channels with inlet/outlet holes was prepared by standard procedure and cured at 80° C. for 7 hours. Prior to the overlay, residual monomeric species were removed from the PDMS by triple overnight washings in hexanes, ethanol and water. The PDMS slab was subsequently clamped to the sensor chip and the metal contact pads were wire bonded to a custom printed circuit board. Upon assembling the microfluidic device, the silicon oxide above the field sensitive region was regenerated by a 30 second etch with 5 μl of buffered oxide etching solution (ammonium fluoride:hydrogen fluoride 7:1 v/v) and a thorough rinse with DI water. The device was then allowed to equilibrate until the baseline signal was stable and long-range drift was insignificant.

Surface Chemistry

Devices were functionalized by physisorption of protomine to the field sensitive region after attachment of the PDMS microfluidic channels. A 20 μM solution of protamine was transported through the channel in a 10 mM P—C buffer for 10 minutes and subsequently rinsed with the buffer. Since only the field sensitive region is sensitive to charge dependent changes in surface potential, it is the only region of the device which absorbs the protamine. A control sensor was prepared in a separate flow channel by the same procedure except that bovine serum albumin (“BSA”) was used in place of protamine.

In other aspects, AT-III was used in place of protamine. In this example, AT-III was covalently attached to the sensor surface by the following procedure:

(1) The freshly prepared sensor chips without the PDMS microfluidics was rinsed with absolute ethanol three times (3×) and incubated with a 1% (v/v) ethanolic solution of propyltrimethoxysilane aldehyde for 20 minutes.

(2) The chip was rinsed three times (3×) with ethanol, incubated in an oven for 30 minutes at 80° C., and rinsed three times (3×) with water.

(3) The chip was overlaid by a split-channel PDMS slab.

(4) The PDMS microfluidics were then overlaid to the chip and the “active” sensor was treated with a 1.0 mg/ml solution of avidin in 100 mM phosphate buffer pH 8.0 containing 50 mM NaCHBH₃ for 3 hours.

(5) Upon rinsing with buffer, the unreacted aldehyde sites were quenched by a similar treatment using 0.5 M ethanolamine instead of avidin.

(6) The device was then treated with a 1.0 mg/ml solution of bAT-III for 6 hours, rinsed three times (3×) with buffer, and allowed to equilibrate.

The “passivated” control sensor was prepared by covalent immobilization of BSA to the sensor surface using the same procedure.

Instrumentation

The surface potential of the filed sensitive region was determined by applying an AC signal (50 mV sine wave at 4 kHz) to the reference electrode and measuring the resulting current through the EIS structure with a current preamp (Keithley Model 428) and lock-in amplifier. The p-type field sensitive region was biased to partial depletion in order to maximize sensitivity to changes in surface potential. The n-type substrate was back biased to 1 V. Capacitance-voltage curves of the EIS structure were acquired in order to determine the optimal p-type bias point. Once biased, the surface potential resolution was ˜10 uV in a 1 Hz bandwidth and the linear range was ˜100 mV. All signals were calibrated by applying a 2.5 mV change in p-type bias potential. Data was acquired with LabView software at 12-bit accuracy with a sampling rate of 10 Hz.

The long-term stability of the surface potential measurement was increased by grounding the channel inlet and outlet with silver wires coated with electrolytically deposited silver chloride. In some aspects, these electrodes were used in place of the on-chip gold reference electrode. In this aspect, the AC signal was applied directly to the silver chloride-coated silver wires.

Chemicals

The following chemicals were used (place of purchase in parentheses):

(1) Heparin sodium from porcine intestinal mucosa (Celsus);

(2) Protamine sulfate from salmon, avidin, biotinylated BSA, chondroitin sulfate, and serum from clotted human male blood (Sigma-Aldrich);

(3) Trimethoxypropylsilane aldehyde (United Chemical Technologies);

(4) Arixtra (Henry Schein, manufactured by Organon, Inc.);

(5) Human antithrombin III (Bayer Corp.).

All buffers were prepared fresh using Nano-pure water and filtered before use. Serum samples, filtered through a 0.2 μm membrane, were diluted with distilled water to a final of 10% (v/v), and they contained 0.05% (w/v) NaN₃ to prevent microbial growth. The running buffer was a 3.0 mM phosphate-citrate buffer containing 7.0 mM NaCl pH 7.0 (total ionic strength 10.0 mM) for aqueous solution measurements and 10% phosphate-buffered saline (“PBS”) for serum measurements.

Biotinylated AT-III was prepared using a previously established method disclosed in Keiser N et al., Nat Med, 2001 January; 7(1):123-8. As disclosed, AT-III was incubated for an hour with excess ardeparin sodium (from Celsus). The protein was then biotinylated with EZ-link sulfo-NHS biotin (from Pierce) as per the manufacturer's instructions. Excess biotin was removed by spin column with a molecular weight cutoff (“MWCO”) of 10,000 (from Millipore). Heparin was removed by five (5) sequential washes with 1M NaCl followed by three (3) washes in water, in a centrifugal filter device (from Millipore) with a MWCO of 10,000. The total protein concentration was 1.2 mg/ml, determined using a Bradford assay. The affinity of the biotinylated protein to heparin was confirmed by SDS-PAGE electrophoresis.

The biotinylated AT-III was incubated with heparin-sepharose beads for 30 minutes. The beads were then washed three times to remove the unbound AT-III. The beads were resuspended in SDS-PAGE sample buffer and were loaded onto a protein gel. The presense of the protein was visualized by a Coomassie stain of the gel. The affinity to streptavidin was also confirmed using SDS-PAGE electrophoresis.

Experimental Setup

For all measurements, solutions were introduced into the device by using a constant-flow fluid delivery system involving an in-line degasser, an HPLC pump, and an autosampler. The analyte exposure times were controlled by adjusting the flow rate (usually 1.0-10.0 μl/min) and the injection volume of the analyte (usually 5.0-40.0 μl). Before and after each analyte injection, the sensor was rinsed thoroughly using “running” buffer identical to that of the analyte solution. Upon each measurement, the surface of the active sensor was regenerated by an incubation for ten (10) minutes with 20 mM protamine solution (for the protamine sensor) or 2.0 M NaCl solution (for the AT-III sensor). The data was processed using Matlab, and the graphs and fitted curves were obtained using SigmaPlot.

What is claimed is:

1. A microfluidic device for real-time detection of heparin, comprising:
at least one field-effect sensor having an electrolyte-insulator-silicon structure, wherein a surface potential of the sensor directly detects heparin.

2. The device of claim 1, wherein the field-effect sensor further comprises:
an active sensing surface;
a control sensing surface; and
at least one microfluidic channel.

3. The device of claim 2, wherein the active sensing surface comprises protamine.

4. The device of claim 2, wherein the active sensing surface comprises antithrombin III.

5. The device of claim 2, wherein the active sensing surface comprises at least one substance exhibiting a high affinity to heparin.

6. The device of claim 2, further comprising a liquid delivery system, wherein the liquid delivery system delivers solutions into the field-effect sensor through the at least one microfluidic channel.

7. The device of claim 6, wherein the liquid delivery system comprises:
an in-line degasser;
an HPLC pump; and
an autosampler.

8. The device of claim 1, comprising a control unit, wherein the control unit controls the environmental conditions of the field-effect sensor.

9. The device of claim 1, comprising a means to measure the surface potential of the electrolyte-insulator-silicon structure.

10. The device of claim 1, comprising a means to transmit the surface potential of the electrolyte-insulator-silicon structure.

11. A method of detecting heparin in real-time, comprising:
binding heparin to the surface of a field-effect sensor, wherein the field-effect sensor comprises an electrolyte-insulator-silicon structure; and
measuring an electrical signal of the electrolyte-insulator-silicon structure.

12. The method of claim 11, comprising exposing the surface of the field-effect sensor to protamine.

13. The method of claim 11, comprising exposing the surface of the field-effect sensor to antithrombin III.

14. The method of claim 11, comprising exposing the surface of the field-effect sensor to at least one substance exhibiting a high affinity to heparin.

15. The method of claim 11, comprising measuring the capacitance of the electrolyte-insulator-silicon structure.

16. The method of claim 11, comprising delivering solutions into the field-effect sensor through a liquid delivery system.

17. The method of claim 11, comprising delivering solutions into the field-effect sensor through at least one microfluidic channel.

18. The method of claim 11, comprising transmitting the electrical signal to a user-interface monitor.

19. The method of claim 11, altering the depth of a carrier depletion region beneath the electrolyte-insulator-silicon structure surface.
